(19) United States
(12) Patent Application Publication
(10) Pub. No.: US 2025/0387062 A1
(43) Pub. Date: Dec. 25, 2025

(54) BIOLOGICAL INFORMATION MEASURING DEVICE

(71) Applicant: OMRON HEALTHCARE Co., Ltd., Kyoto (JP)

(72) Inventor: Kenji ONO, Kyoto (JP)

(21) Appl. No.: 19/305,366

(22) Filed: Aug. 20, 2025

Related U.S. Application Data
(63) Continuation of application No. PCT/JP2023/040737, filed on Nov. 13, 2023.

(30) Foreign Application Priority Data
Mar. 30, 2023 (JP) 2023-056119

(51) Int. Cl. A61B 5/28; A61B 5/00; A61B 5/308

(52) U.S. Cl. CPC A61B 5/28; A61B 5/308; A61B 5/6843

(57) ABSTRACT

A biological information measuring device including a first electrode, a second electrode, and a third electrode, and configured to measure biological information of a measurement target based on a potential difference between the first electrode and the second electrode with a potential of the third electrode used as a reference potential, the biological information measuring device including: a first non-inverting amplifier circuit; a first pull-up resistor; a first converter; a second non-inverting amplifier circuit; a second pull-up resistor; a second converter; a differential amplifier circuit configured to amplify a difference between a first amplified potential amplified and output by the first non-inverting amplifier circuit and a second amplified potential amplified and output by the second non-inverting amplifier circuit, and to output the biological information; and a processor configured to perform processing for measuring the biological information.

### CROSS-REFERENCE TO RELATED APPLICATIONS

This application is the U.S. national stage application filed pursuant to 35 U.S.C. 365(c) and 120 as a continuation of International Patent Application No. PCT/JP2023/040737, filed Nov. 13, 2023, which application claims priority to Japanese Patent Application No. 2023-056119, filed Mar. 30, 2023, which applications are incorporated herein by reference in their entireties.

### TECHNICAL FIELD

The present invention belongs to a technical field related to healthcare, and particularly relates to a biological information measuring device.

### BACKGROUND

In recent years, it has become widespread to perform health management by measuring information on the body and health of an individual such as a blood pressure value and an electrocardiographic waveform (hereinafter, also referred to as biological information) with a measuring device, and recording and analyzing the measurement results with an information terminal.

As an example of the measuring device as described above, a portable electrocardiographic measuring device configured to measure an electrocardiographic waveform immediately when an abnormality, such as pain and palpitation in a chest, occurs in everyday life has been proposed. An early detection of heart disease and a contribution to appropriate treatment are desired (for example, Patent Document 1).

Patent Document 1 discloses a configuration of a portable biological signal telemeter device that acquires an electrocardiogram by three electrodes attached to a subject, the device including an electrode abnormality detection circuit that detects an attachment state of the electrodes to the subject, and outputs an electrode removal signal when an abnormality in the electrode attachment occurs. According to such a configuration, when an abnormality occurs in the attachment state of the electrode, a user can recognize the abnormality and perform reattachment of the electrode, or the like.

### CITATION LIST

### Patent Literature

Patent Document 1: JP H6-269417 A

### SUMMARY

### Technical Problem

According to the technique disclosed in Patent Document 1, when an abnormality in the electrode attachment occurs, the abnormality is notified, and the supply of power to the circuit for electrocardiographic waveform measurement (and transmission of waveform data to the outside) is cut off. However, in practice, the potential of the electrode may vary due to body motion or the like even during the electrocardiographic measurement, and the electrode may be determined to be abnormally attached. In addition, even though the electrode is in contact with the skin, the electrode may be determined not to be normally attached particularly in winter when the skin is likely to be dry. This may cause a problem that the electrocardiographic measurement is interrupted many times in the middle or cannot even start.

In view of the above problems, an object of the present invention is to provide a technique capable of obtaining an electrocardiographic waveform with high accuracy and detecting a contact state of each electrode.

### Solution to Problem

A biological information measuring device according to the present invention adopts the following configurations in order to solve the above problems. That is, a biological information measuring device includes a first electrode, a second electrode, and a third electrode, and is configured to measure biological information of a measurement target based on a potential difference between the first electrode and the second electrode with a potential of the third electrode used as a reference potential.
The biological information measuring device includes: a first non-inverting amplifier circuit including a first amplifier having a non-inverting input terminal to which a potential of the first electrode is input;
a first pull-up resistor connected between the first electrode and the first non-inverting amplifier circuit;
a first converter including the first non-inverting amplifier circuit, and configured to output a signal related to a contact state of the first electrode with the measurement target by using an output signal from the first non-inverting amplifier circuit;
a second non-inverting amplifier circuit including a second amplifier having a non-inverting input terminal to which a potential of the second electrode is input;
a second pull-up resistor connected between the second electrode and the second non-inverting amplifier circuit;
a second converter including the second non-inverting amplifier circuit, and configured to output a signal related to a contact state of the second electrode with the measurement target by using an output signal from the second non-inverting amplifier circuit;
a differential amplifier circuit configured to amplify a difference between a first amplified potential amplified and output by the first non-inverting amplifier circuit and a second amplified potential amplified and output by the second non-inverting amplifier circuit, and to output the biological information; and
a processor configured to perform processing for measuring the biological information.

The resistance value of the pull-up resistor can be, for example, 200 MΩ or more, and more desirably 300 MΩ or more. With the configuration described above, signals indicating the potentials of the first electrode and the second electrode are amplified at the previous stage to the input to the differential amplifier that amplifies and outputs the potential difference between the first electrode and the second electrode. Thus, biological information can be accurately measured using a signal having a high signal (S)/noise (N) ratio. In addition, the pull-up resistor is disposed between the first electrode and the non-inverting amplifier circuit to which the first electrode is connected, and the pull-up resistor is disposed between the second electrode and the non-inverting amplifier circuit to which the second electrode is connected. Thus, noise of a signal indicating a potential of each electrode can be reduced. A signal related to the contact state of each electrode with the measurement target is output using the output signal from the non-inverting amplifier circuit to which the signal described above is input. Thus, a circuit for contact detection is not likely to affect an electrocardiographic waveform, and the measurement of the electrocardiographic waveform with high accuracy and the detection of the contact state of the electrode can be performed in parallel.

In addition, the first contact signal output means may output the signal related to the contact state of the first electrode with the measurement target by using the first amplified potential, and the second contact signal output means may output the signal related to the contact state of the second electrode with the measurement target by using the second amplified potential. Conversely, each contact signal output means may output a signal related to the contact state of its corresponding electrode with the measurement target by using a signal having an amplification factor of 1.

In addition, the biological information measuring device may further include storage configured to store the signal related to the contact state of the first electrode with the measurement target, and the signal related to the contact state of the second electrode with the measurement target at least during processing for measuring the biological information. This allows the contact state of the electrode during the electrocardiographic measurement to be confirmed later.

In addition, each of the first contact signal output means and the second contact signal output means may include an analog (A)/digital (D) converter, and the biological information measuring device may further include contact state digital signal output configured to classify the contact state of each of the first electrode and the second electrode with the measurement target into at least three levels by using a digital signal output from the A/D converter. In addition, information on the classified contact state at least during the processing for measuring the biological information may be stored in the storage.

A threshold for the classification of a digitized signal can be appropriately set by a user based on contact resistance, electrocardiographic recording quality, and the like. According to this configuration, the user can confirm a contact state classified into different levels according to the contact level, not as an analog value, and easily ascertain the contact state of an electrode.

### Advantageous Effects of Invention

The present invention can provide a biological information measuring device capable of obtaining an electrocardiographic waveform with high accuracy and detecting a contact state of each electrode.

### BRIEF DESCRIPTION OF DRAWINGS

Various embodiments are disclosed, by way of example only, with reference to the accompanying schematic drawings in which corresponding reference symbols indicate corresponding parts, in which:

FIG. 1(A) is a front view illustrating a configuration of a portable electrocardiographic measuring device according to an embodiment;

FIG. 1(B) is a rear view illustrating the configuration of the portable electrocardiographic measuring device according to the embodiment;

FIG. 1(C) is a left side view illustrating the configuration of the portable electrocardiographic measuring device according to the embodiment;

FIG. 1(D) is a right side view illustrating the configuration of the portable electrocardiographic measuring device according to the embodiment;

FIG. 1(E) is a plan view illustrating the configuration of the portable electrocardiographic measuring device according to the embodiment;

FIG. 1(F) is a bottom view illustrating the configuration of the portable electrocardiographic measuring device according to the embodiment;

FIG. 2 is a block diagram for explaining a functional configuration of the portable electrocardiographic measuring device according to the embodiment;

FIG. 3 is a circuit diagram illustrating a part of an electric circuit configuration of the portable electrocardiographic measuring device according to the embodiment;

FIG. 4(A) is a first diagram illustrating the relationship between a pull-up resistance value and an electrocardiographic waveform of the portable electrocardiographic measuring device according to the embodiment;

FIG. 4(B) is a second diagram illustrating the relationship between a pull-up resistance value and an electrocardiographic waveform of the portable electrocardiographic measuring device according to the embodiment;

FIG. 4(C) is a third diagram illustrating the relationship between a pull-up resistance value and an electrocardiographic waveform of the portable electrocardiographic measuring device according to the embodiment;

FIG. 4(D) is a fourth diagram illustrating the relationship between a pull-up resistance value and an electrocardiographic waveform of the portable electrocardiographic measuring device according to the embodiment;

FIG. 5 is an explanatory diagram for explaining an experimental example of the portable electrocardiographic measuring device according to the embodiment;

FIG. 6 is a flowchart showing an example of the flow of processing related to electrocardiographic measurement performed by the portable electrocardiographic measuring device according to the embodiment; and,

FIG. 7 is a circuit diagram illustrating a part of an electric circuit configuration of a portable electrocardiographic measuring device according to a modified example.

### DESCRIPTION OF EMBODIMENTS

### First Embodiment

Specific embodiments of the present invention are described below with reference to the drawings. It should be noted that the dimensions, material, shape, relative arrangement and the like of the constituent components described in the embodiments are not intended to limit the scope of this invention to them alone, unless otherwise stated.

### Electrocardiographic Measuring Device

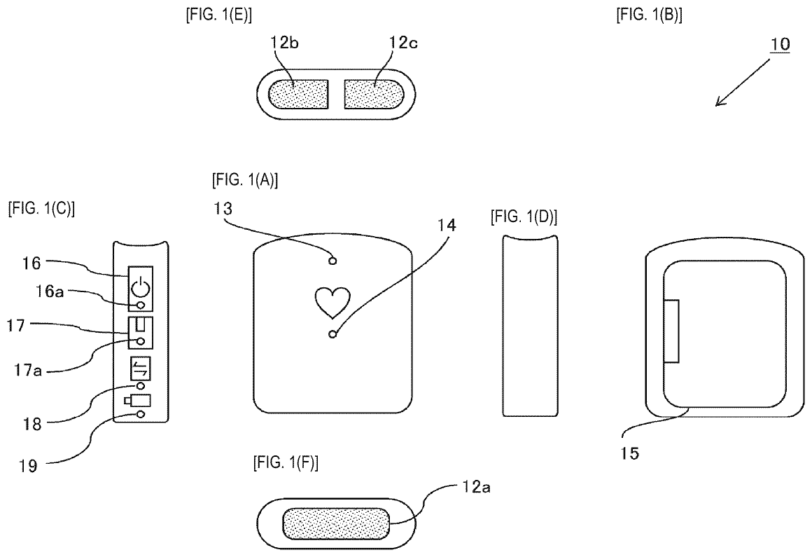

FIG. 1 is a diagram illustrating a configuration of a portable electrocardiograph 10 according to the present embodiment. FIG. 1(A) is a front view illustrating the front of a body. Similarly, FIG. 1(B) is a rear view. FIG. 1(C) is a left side view. FIG. 1(D) is a right side view. FIG. 1(E) is a plan view. FIG. 1(F) is a bottom view.

A bottom surface of the portable electrocardiograph 10 is provided with a left electrode 12a brought into contact with the left side of the body during electrocardiographic measurement. Similarly, a top surface side of the portable electrocardiograph 10, opposite to the bottom surface, is provided with a first right electrode 12b brought into contact with the middle phalanx of a right-hand index finger, and a second right electrode 12c brought into contact with the proximal phalanx of the right-hand index finger.

During electrocardiographic measurement, the portable electrocardiograph 10 is held by the right hand, and the right-hand index finger is positioned on the top surface portion of the portable electrocardiograph 10 in proper contact with the first right electrode 12b and the second right electrode 12c. The left electrode 12a is then brought into contact with a skin at a location corresponding to a desired measurement method. For example, when the measurement is performed by a so-called I induction, the left electrode 12a is brought into contact with the palm of the left hand, and when the measurement is performed by a so-called V4 induction, the left electrode 12a is brought into contact with the skin slightly leftward in the epigastric region of the left chest and below the nipple.

In addition, various operation units and indicators are disposed on a left side surface of the portable electrocardiograph 10. Specifically, a power switch 16, a power LED 16a, a Bluetooth (registered trademark) low energy (BLE) communication button 17, a BLE communication LED 17a, a memory residual display LED 18, a battery change LED 19, and the like, are provided.

In addition, a measurement state notification LED 13 and an analysis result notification LED 14 are provided on a front surface of the portable electrocardiograph 10, and a battery housing opening and a battery cover 15 are disposed at a rear surface of the portable electrocardiograph 10

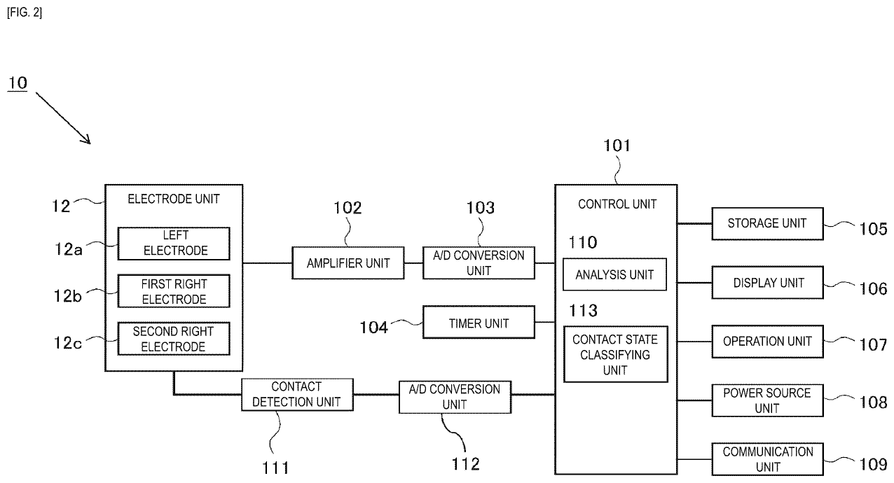

FIG. 2 is a block diagram illustrating a functional configuration of the portable electrocardiograph 10. As illustrated in FIG. 2, the portable electrocardiograph 10 includes functional units: a control unit 101, an electrode unit 12, an amplifier unit 102, an A/D conversion unit 103, a timer unit 104, a storage unit 105, a display unit 106, an operation unit 107, a power source unit 108, a communication unit 109, a contact detection unit 111, and an A/D conversion unit 112.

The control unit 101 manages the control of the portable electrocardiograph 10, and includes a central processing unit (CPU), for example. Upon receiving a user's operation via the operation unit 107, the control unit 101 controls each component of the portable electrocardiograph 10 to perform various types of processing such as electrocardiographic measurement and information communication in accordance with a predetermined recording medium. Note that the predetermined recording medium is stored in the storage unit 105 to be described below and is read therefrom.

In addition, the control unit 101 includes, as functional modules, an analysis unit 110 for analyzing electrocardiographic waveforms, and a contact state classifying unit 113. The analysis unit 110 analyzes a measured electrocardiographic waveform for the presence or absence of waveform disturbance, or the like, and outputs a result indicating whether the electrocardiographic waveform obtained at least during the measurement is normal. The contact state classifying unit 113 classifies the contact states of the left electrode 12a and the first right electrode 12b detected by the contact detection unit 111 into four levels. The detection of the contact state and the level classification thereof are described below.

The electrode unit 12 includes the left electrode 12a, the first right electrode 12b, and the second right electrode 12c, and functions as a sensor for detecting an electrocardiographic waveform. Specifically, the second right electrode 12c is used as a ground (GND) electrode, and the potential difference between a potential of the left electrode 12a and a potential of the first right electrode 12b with respect to the reference potential of the ground (GND) electrode is continuously measured, thereby acquiring the electrocardiographic waveform. A specific circuit configuration for the electrocardiographic waveform detection is described below.

The amplifier unit 102 has a function of amplifying a signal indicating the electrocardiographic waveform output from the electrode unit 12 as described below. The A/D conversion unit 103 has a function of converting an analog signal amplified by the amplifier unit 102 into a digital signal, and transmitting the digital signal to the control unit 101.

The timer unit 104 has a function of measuring time with reference to a real time clock (RTC). For example, as described below, when electrode contact detection processing is performed, the timer unit 104 counts the time during which all of the left electrode 12a, the first right electrode 12b, and the second right electrode 12c are in contact with the body. In addition, during the electrocardiographic measurement, the timer unit 104 may count the time until the end of the measurement, and output the counted time.

The storage unit 105 includes a main storage device such as a random access memory (RAM) and a read only memory (ROM), and stores various kinds of information such as an application recording medium, a measured electrocardiographic waveform, and an analysis result. In addition to the RAM and the ROM, the storage unit 105 includes, for example, a long-term storage medium such as a flash memory.

The display unit 106 includes the measurement state notification LED 13, the analysis result notification LED 14, the power LED 16a, the BLE communication LED 17a, the memory residual display LED 18, the battery change LED 19, and the like, and transmits the state of the device to the user by lighting or blinking the LEDs. In addition, the operation unit 107 includes the power switch 16, the communication button 17, and the like, and has a function of receiving an input operation from the user and causing the control unit 101 to perform processing according to the operation.

The power source unit 108 includes a battery that supplies power required for operating the device. The battery may be, for example, a secondary battery such as a lithium ion battery, or a primary battery.

The communication unit 109 includes an antenna for wireless communication, and has a function of communicating with another device such as an information processing terminal by at least BLE communication. In addition, the communication unit 109 may include a terminal for wired communication.

The contact detection unit 111 includes an electric circuit connected to the left electrode 12a and the first right electrode 12b, detects a contact state between the left electrode 12a and the skin surface of a measurement target and a contact state between the first right electrode 12b and the skin surface of a measurement target, and outputs a signal according to the level of the contact state. The A/D conversion unit 112 converts an analog signal output by the contact detection unit 111 into a digital signal and outputs the digital signal to the control unit 101.

### Electric Circuit Configuration

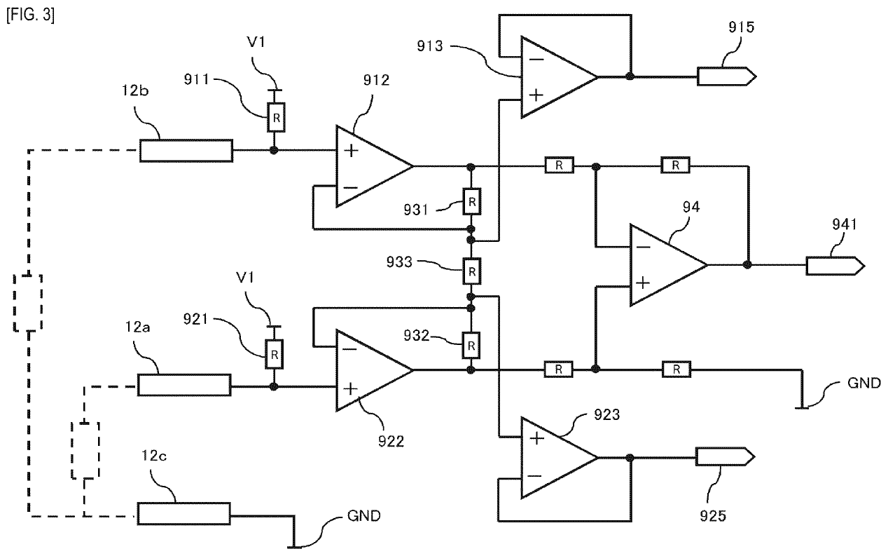

The contact state detection and the electrocardiographic waveform measurement in the portable electrocardiograph 10 according to the present embodiment are described below with reference to FIG. 3. FIG. 3 is a circuit diagram illustrating an outline of an electric circuit including the electrodes of the portable electrocardiograph 10.

As illustrated in FIG. 3, the second right electrode 12c is connected to the reference potential GND, and functions as a ground electrode. The first right electrode 12b is connected to a power source potential V1 via a right pull-up resistor 911. The left electrode 12a is connected to the power source potential V1 via a left pull-up resistor 921. The power source potential V1 is set to a potential (for example, 4 V) that is higher than the reference potential GND and can secure a sufficient bias.

Therefore, when the power source is in an ON state, and both the first right electrode 12b and the second right electrode 12c are properly in contact with the skin of the body, a current flows through the impedance of the human body to the second right electrode 12c having a lower potential than the first right electrode 12b, and the potential of the first right electrode 12b varies. Such a variation in the potential depends on the contact state between the first right electrode 12b (and the second right electrode 12c) and the skin surface.

That is, since the potential becomes lower as the first right electrode 12b is more firmly in contact with the skin, the contact state between the first right electrode 12b and the skin can be determined based on the potential. The same applies to the left electrode 12a. Note that the circuit indicated by the dashed line portion in FIG. 3 indicates the path of the current via the impedance of the human body.

The right pull-up resistor 911 and the left pull-up resistor 921 are set to a resistance value high enough to secure the accuracy of an electrocardiographic waveform to be detected (for example, 200 MΩ, desirably 300 MΩ or more). The relationship between the electrocardiographic waveform and the value of the pull-up resistor is described in detail below.

In the circuit illustrated in FIG. 3, five amplifiers are disposed, including a right non-inverting amplifier 912, a right buffer amplifier 913, a left non-inverting amplifier 922, a left buffer amplifier 923, and a differential amplifier 94.

As illustrated in FIG. 3, the potential of the first right electrode 12b is input to a + input terminal of the right non-inverting amplifier 912. Subsequently, a right amplified signal amplified by an amplification factor defined by a first amplification factor determining resistor 931 and a third amplification factor determining resistor 933 is output from an output terminal of the right non-inverting amplifier 912 and input to a − terminal of the differential amplifier 94. On the other hand, a signal having the same potential as the potential input to the + input terminal of the right non-inverting amplifier 912 is input to a + input terminal of the right buffer amplifier 913 via the right non-inverting amplifier 912. That is, the right non-inverting amplifier 912 functions as a normal amplifier (signal amplifier), and also functions as a buffer (voltage follower).

The right buffer amplifier 913 functions as a buffer, and a signal having the same potential as the potential input to the + input terminal is output from an output terminal. The output signal is input to the A/D conversion unit 112 as a right contact state signal 915, is converted into a digital signal, and is transmitted to the control unit 101. That is, the right buffer amplifier 913 of the present embodiment is included in first contact signal output means according to the present invention.

The potential of the left electrode 12a is input to a + input terminal of the left non-inverting amplifier 922. Subsequently, a left amplified signal amplified by an amplification factor defined by a second amplification factor determining resistor 932 and the third amplification factor determining resistor 933 is output from an output terminal of the left non-inverting amplifier 922, and input to a + terminal of the differential amplifier 94. On the other hand, a signal having the same potential as the potential input to the + input terminal of the left non-inverting amplifier 922 is input to a + input terminal of the left buffer amplifier 923 via the left non-inverting amplifier 922. That is, the left non-inverting amplifier 922 also functions as a normal amplifier, and also functions as a buffer, similarly to the right non-inverting amplifier 912. The resistance values of the first amplification factor determining resistor 931 and the second amplification factor determining resistor 932 are set to the same value.

The left buffer amplifier 923 functions as a buffer, and a signal having the same potential as the potential input to the + input terminal is output from an output terminal. The output signal is input to the A/D conversion unit 112 as a left contact state signal 925, is converted into a digital signal, and is transmitted to the control unit 101. That is, the left buffer amplifier 923 of the present embodiment is included in second contact signal output means according to the present invention.

The differential amplifier 94 is a differential amplifier that amplifies and outputs the difference between the potential of the first right electrode 12b input to the − input terminal and amplified and output by the right non-inverting amplifier 912 and the potential of the left electrode 12a input to the + input terminal and amplified and output by the left non-inverting amplifier 922. That is, the differential amplifier 94 is included in the amplifier unit 102, and the signal output from the differential amplifier 94 is an electrocardiographic signal of the measurement target. The electrocardiographic signal is further input to the A/D conversion unit 103, and the signal converted into a digital signal is transmitted to the control unit 101 and recorded as an electrocardiographic waveform in the storage unit 105 by the control unit 101.

As described above, since the signals input to the differential amplifier 94 have already been amplified by the right non-inverting amplifier 912 and the left non-inverting amplifier 922 before the input stage, signals having a high S/N ratio can be used for electrocardiographic measurement, and the tolerance ability to electromagnetic noise and the like can be improved.

### Experimental Example Regarding Pull-Up Resistance Value

When body motion is produced during the electrocardiographic measurement, since the body resistance and the contact resistance between the electrode and the skin are changed, the potential of each electrode varies (this phenomenon significantly appears during the electrocardiographic measurement particularly in winter when the skin is likely to be dry). Therefore, the pull-up resistance value is desirably set to a value of several hundred megaohms which is so high as not to be affected by the body motion. The results of an experimental example regarding pull-up resistance values are described below.

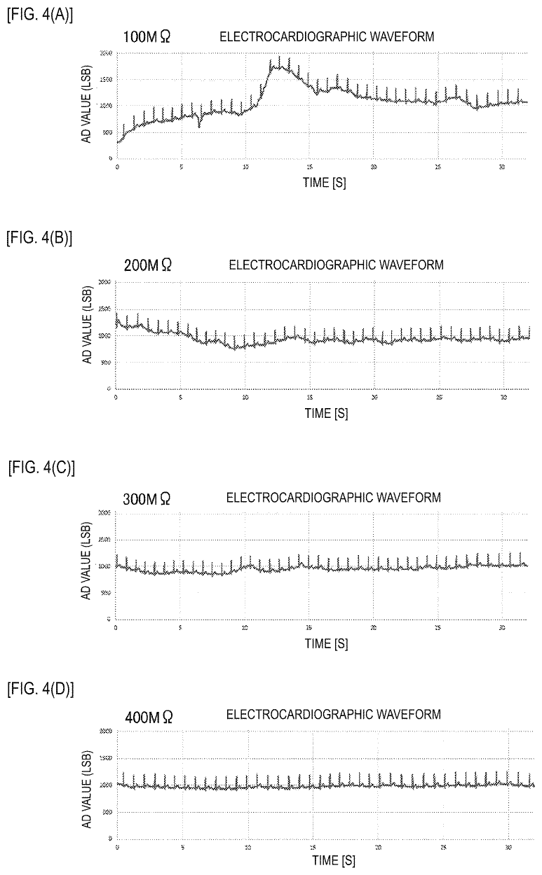

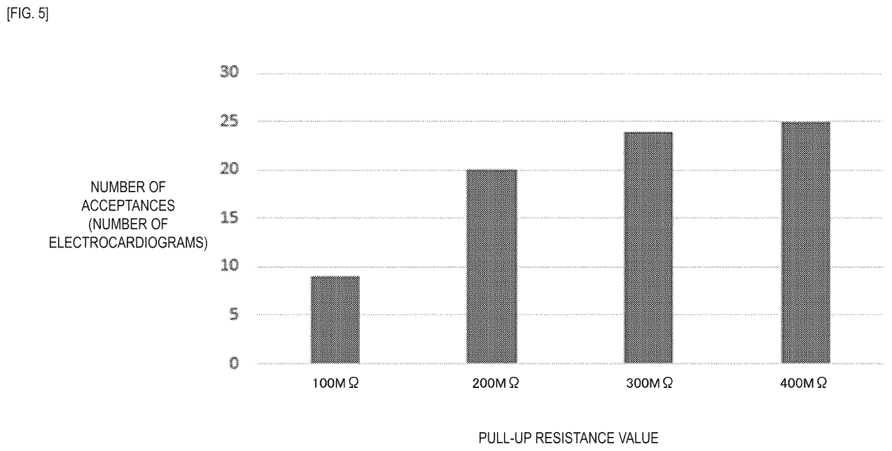

In the experiments according to the present experimental example, the electrocardiogram measurement (recording) was performed with the pull-up resistance values set to 100 MΩ, 200 MΩ, 300 MΩ, and 400 MΩ. The results are illustrated in FIGS. 4(A) to 4(D) and FIG. 5. FIG. 4(A) illustrates an example of an electrocardiographic waveform measured when the pull-up resistance value is 100 MΩ. FIG. 4(B) illustrates an example of an electrocardiographic waveform measured when the pull-up resistance value is 200 MΩ. FIG. 4(C) illustrates an example of an electrocardiographic waveform measured when the pull-up resistance value is 300 MΩ. FIG. 4(D) illustrates an example of an electrocardiographic waveform measured when the pull-up resistance value is 400 MΩ. FIG. 5 is a graph showing the number of electrocardiographic waveforms satisfying an acceptance/rejection criterion regarding the stability of a baseline among the electrocardiographic waveforms measured at the respective resistance values of 100 MΩ, 200 MΩ, 300 MΩ, and 400 MΩ.

As illustrated in FIGS. 4(A) to 4(D), it is understood that the electrocardiographic waveform is measured more accurately as the pull-up resistance value increases. In particular, when the resistance value is 100 MΩ, a state in which the baseline of the electrocardiogram is greatly varied can be read. On the other hand, when the resistance value is set to 400 MΩ, a state in which such a variation in the baseline is not observed and the waveform is stable is read.

The graph illustrated in FIG. 5 shows how many electrocardiographic waveforms out of 26 electrocardiographic waveforms for each resistance value are acceptable, based on the acceptance/rejection criterion of whether the electrocardiographic waveform falls within a range of ±500 least significant bits (LSB) with respect to an AD value, 1024 LSB, serving as the center of the electrocardiographic baseline. At the pull-up resistance value of 100 MΩ, only nine electrocardiographic waveforms, which are less than half of the 26 electrocardiographic waveforms, are acceptable, whereas at the pull-up resistance values of 200 MΩ or more, 20 or more electrocardiographic waveforms satisfy the acceptance criterion. Therefore, the pull-up resistance value set to 200 MΩ or more is considered to be desirable. Moreover, in view of the number of acceptable electrocardiographic waveforms in the cases of 200 MΩ, 300 MΩ, and 400 MΩ, the pull-up resistance value of 300 MΩ or more is more desirable.

### Information Stored in Storage Unit

The contact state classifying unit 113 that is a functional module of the control unit 101 classifies the level of the respective contact states of the first right electrode 12b and the left electrode 12a with the skin into four levels, “good contact”, “slightly poor contact”, “poor contact”, and “no contact”, by using the right contact state signal 915 and the left contact state signal 925 digitally converted by the A/D conversion unit 112. When the right contact state signal 915 and the left contact state signal 925 vary with time, the classified level of the contact state is affected by this variation. Accordingly, the level indicating the contact state also varies. Therefore, information indicating the classified level of the contact state is recorded in the storage unit 105 as time-series data.

The measured electrocardiographic waveform and the classified level of the contact state are stored in the storage unit 105 in association with information on the time at which each of the measured electrocardiographic waveform and the classified level of the contact state is acquired. Therefore, the electrocardiographic waveform and the level of the contact state can be used in synchronization with each other. For example, when the stored electrocardiographic waveform is displayed on a display later for confirmation, the electrocardiographic waveform and the levels of the respective contact states of the first right electrode 12b and the left electrode 12a when this electrocardiographic waveform is detected can be confirmed together.

The analysis unit 110 analyzes the measured electrocardiographic waveform for the presence or absence of waveform disturbance, or the like, and outputs a result indicating whether the electrocardiographic waveform obtained at least during measurement is normal. The analysis result is notified to the user by lighting or blinking the analysis result notification LED 14, and is recorded in the storage unit 105. By using the information stored in this manner, the information of the analysis result by the analysis unit 110 and the information related to the measurement time can be displayed in association with the information of the electrocardiographic waveform and the contact level. This can further improve the convenience for the user.

### Electrocardiographic Measurement Processing Using Portable Electrocardiograph

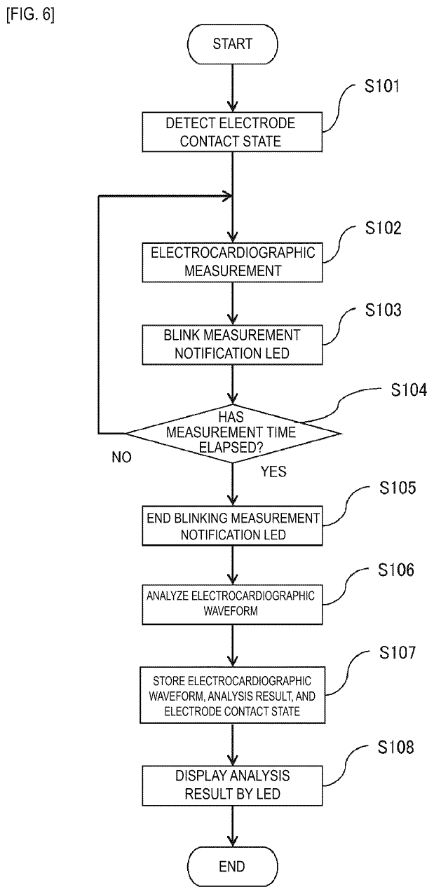

An operation of the portable electrocardiograph 10 when the electrocardiographic measurement is performed is described below. FIG. 6 is a flowchart showing a processing procedure when the electrocardiographic measurement is performed using the portable electrocardiograph 10.

Referring to FIG. 6, prior to the measurement, the user first operates the power switch 16 to turn on the power source of the portable electrocardiograph 10. As a result, the power LED 16a is lighted to indicate that the power source is on. Subsequently, the user holds the portable electrocardiograph 10 with the right hand, with the right-hand index finger in contact with the first right electrode 12b and the second right electrode 12c, and with the left electrode 12a in contact with the skin at a location to be measured. As a result, the control unit 101 starts detecting the contact state of each of the electrodes and classifying the contact level via the electrode unit 12 and the contact detection unit 111 (S101).

Subsequently, the control unit 101 performs electrocardiographic measurement processing (S102). While the electrocardiographic measurement is performed, the control unit 101 stores a measured value in the storage unit 105 at any time, and indicates that the electrocardiographic measurement is being performed by blinking the measurement state notification LED 13 on the front surface of the body in a predetermined rhythm (S103).

Subsequently, the control unit 101 performs processing for determining whether a predetermined measurement time (for example, 30 seconds) for the electrocardiographic measurement has elapsed (step S104). When it is determined that the predetermined measurement time has not elapsed, the control unit 101 returns to step S102 and repeats the subsequent processing. On the other hand, when it is determined that the predetermined measurement time has elapsed, the control unit 101 ends the measurement and performs processing for ending the blinking of the measurement state notification LED 13 (step S105).

Subsequently, the analysis unit 110 of the control unit 101 analyzes measured data (electrocardiographic waveform) stored in the storage unit 105 (S106). The analysis result is stored in a long-term storage medium along with the electrocardiographic waveform and a classified contact state level (S107). Subsequently, the control unit 101 displays the analysis result by the analysis result notification LED 14 (S108), and ends the series of processing. Note that for the display of the analysis result, the LED may be lighted only when the electrocardiographic waveform is found abnormal, or the LED may be lighted by a lighting and blinking method according to the analysis result.

In the portable electrocardiograph 10 according to the present embodiment and having the above-described configuration, a user can start the measurement without performing any operation other than bringing an electrode into contact with a measurement site after operating the power switch 16. In addition, the pull-up resistors (connected to the bias potential) are disposed between the first right electrode 12b and its corresponding amplifier for buffering and amplification and between the left electrode 12a and its corresponding amplifier for buffering and amplification. Thus, noise of signals indicating the potentials of the respective electrodes can be reduced. Thus, even though the detection of the electrode contact state, and the electrocardiographic measurement are performed at the same time, a highly accurate electrocardiographic waveform can be obtained using a signal having a high S/N ratio.

In addition, the contact states of the first right electrode 12b and the left electrode 12a with the measurement target can be classified into different levels and recorded in synchronization with the electrocardiographic waveform. Therefore, when the electrocardiographic waveform is output to a display or the like and confirmed later, the level indicating the contact state of each electrode when the electrocardiographic waveform is detected can be confirmed together with the electrocardiographic waveform.

### MODIFIED EXAMPLE

The description of the above embodiment is merely illustrative of the present invention, and the present invention is not limited to the specific embodiment described above. Within the scope of the technical idea of the present invention, various modifications and combinations can be made.

For example, in the electric circuit according to the above embodiment, a signal indicating the potential of the first right electrode 12b is input to the right buffer amplifier 913 via the right non-inverting amplifier 912 and then output to the A/D conversion unit 112 for the contact state detection. However, the right buffer amplifier 913 is not necessarily provided for the contact state detection.

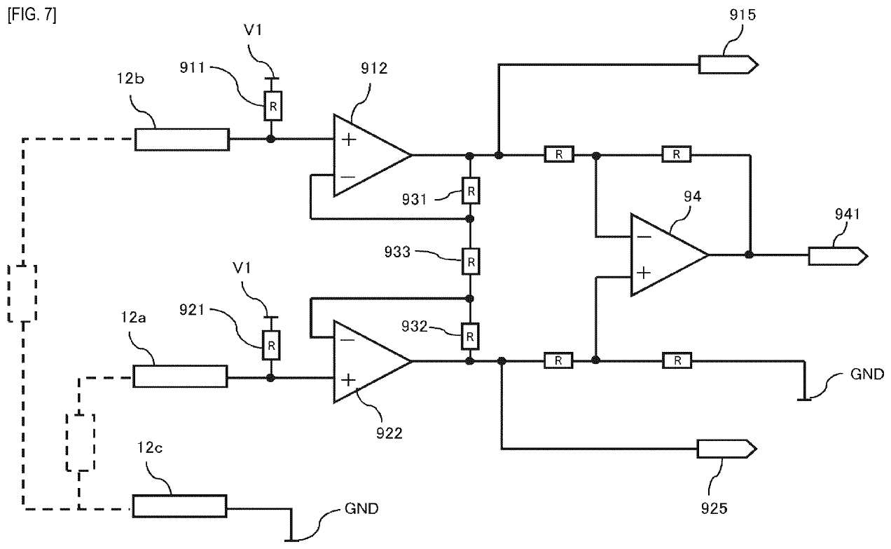

FIG. 7 illustrates an electric circuit diagram according to this modified example. As illustrated in FIG. 7, a signal indicating the potential of the first right electrode 12b is amplified by the right non-inverting amplifier 912 and output, and then the amplified signal is input to the input terminal of the differential amplifier 94 and output as the right contact state signal 915 as it is. The same applies to the circuit for detecting the contact state of the left electrode 12a.

Although not illustrated in the drawing, a modified example is also possible in which the right buffer amplifier 913 is omitted, and a signal having the same potential as the potential of the − input terminal of the right non-inverting amplifier 912 (as in the circuit diagram of the embodiment illustrated in FIG. 3) is output as the right contact state signal 915 as it is. The same applies to the circuit for detecting the contact state of the left electrode 12a.

### OTHER POINTS

In addition, the above embodiment has a configuration in which the right contact state signal 915 and the left contact state signal 925 are output to the A/D conversion unit 112. However, the configuration is not necessarily limited thereto and for example, may be a configuration in which the right contact state signal 915 and the left contact state signal 925 are output to a comparator for determining the contact state in two levels. Although not described in detail in the above embodiment, the electrocardiograph and another information terminal device can be used in cooperation with each other by the BLE communication function of the communication unit 109. Conversely, an electrocardiograph not including the communication function or the LED display unit can also be used.

Although the present invention is applied to a portable electrocardiograph in the above descriptions, the present invention can also be applied to a non-portable electrocardiograph, and can also be applied to biological information measuring devices other than the electrocardiographs.

### REFERENCE NUMERALS LIST

10 Portable electrocardiograph
12a Left electrode
12b First right electrode
12c Second right electrode
13 Measurement state notification LED
14 Analysis result notification LED
15 Battery cover
16 Power switch
16a Power LED
17 Communication button
17a BLE Communication LED
18 Memory residual display LED
19 Battery change LED
911 Right pull-up resistor
912 Right non-inverting amplifier
913 Right buffer amplifier
915 Right contact state signal
921 Left pull-up resistor
922 Left non-inverting amplifier
923 Left buffer amplifier
925 Left contact state signal
931 First amplification factor determining resistor
932 Second amplification factor determining resistor
933 Third amplification factor determining resistor
94 Differential amplifier
941 Electrocardiographic signal
GND Reference potential
V1 Power source potential

1. A biological information measuring device including a first electrode, a second electrode, and a third electrode, and configured to measure biological information of a measurement target based on a potential difference between the first electrode and the second electrode with a potential of the third electrode used as a reference potential, the biological information measuring device comprising:
a first non-inverting amplifier circuit including a first amplifier having a non-inverting input terminal to which a potential of the first electrode is input;
a first pull-up resistor connected between the first electrode and the first non-inverting amplifier circuit;
a first converter including the first non-inverting amplifier circuit, and configured to output a signal related to a contact state of the first electrode with the measurement target by using an output signal from the first non-inverting amplifier circuit;
a second non-inverting amplifier circuit including a second amplifier having a non-inverting input terminal to which a potential of the second electrode is input;
a second pull-up resistor connected between the second electrode and the second non-inverting amplifier circuit;
a second converter including the second non-inverting amplifier circuit, and configured to output a signal related to a contact state of the second electrode with the measurement target by using an output signal from the second non-inverting amplifier circuit;
a differential amplifier circuit configured to amplify a difference between a first amplified potential amplified and output by the first non-inverting amplifier circuit and a second amplified potential amplified and output by the second non-inverting amplifier circuit, and to output the biological information; and
a processor configured to perform processing for measuring the biological information, wherein
each of the first converter and the second converter includes an A/D converter, and
the biological information measuring device further comprises contact state digital signal output configured to classify the contact state of each of the first electrode and the second electrode with the measurement target into at least three levels by using a digital signal output from the A/D converter.

2. The biological information measuring device according to claim 1, wherein
the first converter outputs the signal related to the contact state of the first electrode with the measurement target by using the first amplified potential, and
the second converter outputs the signal related to the contact state of the second electrode with the measurement target by using the second amplified potential.

3. The biological information measuring device according to claim 1, further comprising storage configured to store the signal related to the contact state of the first electrode with the measurement target, and the signal related to the contact state of the second electrode with the measurement target at least during processing for measuring the biological information.

4. The biological information measuring device according to claim 1, further comprising storage configured to store information on the classified contact state at least during processing for measuring the biological information.

5. The biological information measuring device according to claim 1, wherein the first pull-up resistor and the second pull-up resistor both have a resistance value of 200 MΩ or more.

6. The biological information measuring device according to claim 5, wherein the first pull-up resistor and the second pull-up resistor both have a resistance value of 300 MΩ or more.
